(19) United States
(12) Patent Application Publication
(10) Pub. No.: US 2020/0383694 A1
(43) Pub. Date: Dec. 10, 2020

(54) Forward Pulling Sculpted Edge Tongue Scraper with Dental Flosser and Dental Pick

(71) Applicants: Hanna Jun, Snoqualmie, WA (US); Jae Jun, Snoqualmie, WA (US)

(72) Inventors: Hanna Jun, Snoqualmie, WA (US); Jae Jun, Snoqualmie, WA (US)

(73) Assignee: Open Arms Holdings LLC, Snoqualmie, WA (US)

(21) Appl. No.: 16/432,996

(22) Filed: Jun. 6, 2019

(51) Int. Cl. A61B 17/24; A61C 15/02; A61C 15/04

(52) U.S. Cl. CPC A61B 17/244; A61C 15/046; A61C 15/02

(57) ABSTRACT

An oral hygiene device for cleaning the surface of a tongue, cleaning between teeth, and massaging the gums at the tooth gum interface. The device comprising: a handle; a continuous sculpted edge on one end of the handle at a 90-degree angle to the handle; a cutout near the end of the handle with the continuous sculpted edge tongue cleaner; a dental flosser with two tines on the other end of the handle. In the handle in the middle is a swing out tapered end floss pick, that rotates away from the handle for use, pivoting away from the handle on a pair of divots on either side of the floss pick, when the handle is twisted.

### RELATED PATENT APPLICATIONS

Application Ser. No. 16/432,991 Parallel to Face Sculpted Edge Tongue Scraper with Dental Flosser and Dental Floss Pick

### BACKGROUND—PRIOR ART

### US Patents

| Pat. No. | Kind Code | Issue Date | Patentee |
|---|---|---|---|
| 6,152,939 | A | Nov. 28, 2000 | Thuy B. Ngyen |
| 6,273,719 | B1 | Aug. 14, 2001 | Nathan Lane Whitman |
| 6,363,949 | B1 | Apr. 2, 2002 | Thomas W. Brown |
| 6,440,140 | B1 | Aug. 27, 2002 | Dasan Potti |

### US Patent Applications

| Publication # | Kind Code | Pub. Date | Applicant |
|---|---|---|---|
| 20040040572 | A1 | Mar. 4, 2004 | Ingram S, Chodrow |
| 20080185016 | A! | Aug. 7, 2008 | John M. Jansheski |
| | | | Lex Shankle |

### 1. Field of the Invention

This invention is in the field of dental hygiene devices, including dental flossers, tongue scrapers and dental picks.

### 2. Background of the Invention Field

Most people brush their teeth perhaps once a day, floss between their teeth perhaps twice a week, and clean their tongue occasionally. Another dental hygiene tool used sporadically is a dental pick

Diligent people brush their teeth one or more times a day and floss at least once a day and frequently scrap their tongues. People who diligent about their dental care also use a dental plastic floss pick to clean between the teeth and also to gently press against the gum. Using the dental plastic floss picks with an in and out motion to remove food particles, plaque and stimulate gums. While dental flossers and tongue scrapers have been combined, the applicant did not discover a three-in-product in searches.

The invention of this application combines flossing, tongue scraping and the use of a plastic dental floss pick into one tool. The major important advancement is the use of a continuous sculpted edge on the tongue scraper, which creates a ruffled effect on the edge for tongue scraping. Those ruffles are what makes the device of this application effective to scrape the tongue to remove the white film from a dirty tongue. A straight edge of traditional tongue scrapers only glides over the surface of the tongue and does not remove the white film effectively. The ruffled edges act like a comb to dig into the surface and which results in this device's effectiveness.

### DESCRIPTION OF PRIOR ART

Prior art includes products with a combination of dental flossers and tongue scrapers, with at least one tongue scraper have an abrasive surface on the edge to improve efficacy, but it is not nearly the same as this device's sculpted edge that creates the ruffle effect. The patents closest to the concept of this device are listed below.
1. U.S. Pat. No. 7,997,287, A dental flosser having a tongue cleaner, a rugous surface on at least one of the arms suitable for being scraped against the tongue of a user for effectively cleaning the tongue, This device does not have a pick, and also the key feature is a rugous surface on the tongue scraper. A rugous surface is defined as a having a rough, ridged, or wrinkled surface. This is not the same as this device which as a sculpted scraper which creates a furled effect which acts like a comb, ruffling the surface. One embodiment in the patent mentions having the rugous surface on raised ridges, but it does not state that the ridges are continuous, and the ridge height is not defined. The sculpted surface of the tongue scraper is much taller than a rugous surface and offers a much stronger comb like effect.
2. US 20040040572 offers a combination tongue scraper and dental flosser, with the dental flosser on one end and the tongue scraper on the other, and a handle in between. This device uses a standard smooth edge tongue scraper and does not have a dental pick.
3. U.S. Pat. No. 6,152,939 is a tongue scraper only, that functions parallel to the face where both ends are positioned near the corner of the mouth, the tongue is stuck out, and the tongue scraper pulled forward. The scraping portion of the product is a flexible strip, with abrasions on it, with the flexible strip having handles on either end.
4. U.S. Pat. No. 627,719 is a toothbrush and tongue scraper combination, with the two on either end of a handle. The tongue scraper is a continuous loop integrally formed with the handle at the second end of the handle and having an edge suitable for scraping the tongue. The tongue scraper in this patent has a traditional smooth edge available in most tongue scrapers today.
5. U.S. Pat. No. 6,363,949 is a combination tongue scraper and dental flosser. The tongue scraper here is a traditional loop with a smooth edge. The innovative feature is that the device containers a dental floss dispenser in addition to a dental flosser.
6. U.S. Pat. No. 6,440,149 is a dental flosser and tongue scrubber combination with the dental flosser and tongue scraper at opposite ends of the handle. In this case the innovative feature in the tongue scraper. The tongue scraper first has two smooth scraper with one being on the outer edge of the device, and one slightly inward of the outer scraper. The disclosed device has two cutouts, but their purpose is uncertain, and the cutouts are not mentioned in the claims.

### SUMMARY OF THE INVENTION

A tongue and tooth cleaning device for cleaning the surface of a tongue, cleaning between teeth, and massaging the gums at the tooth gum interface comprising a handle, a continuous sculpted edge on one end of the handle, at a 90-degree angle to the handle, and a cutout near the end of the handle with the continuous sculpted edge tongue cleaner, a dental flosser with two tines for the floss on the other end of the handle. In the handle in the middle is a swing out tapered end floss pick, that rotates away from the handle when the handle is twisted away from the handle plane, the divots also can be easily broken off allowing dental floss pick to be removed when desired.

### OPERATION OF THE INVENTION

The dental flosser operates conventionally. The tongue scraper works primarily by the person placing the tongue scraper on the tongue and then pulling the tongue scraper forward. The sculpted edge of the tongue scraper asks like a comb to kick out debris from the tongue, some of which can exit the device through a cutout just in front of the tongue scraper. The dental flosser and dental floss pick work conventionally, with the dental flosser pushing floss between the teeth and the floss pick also use to remove debris between the teeth but additionally being used to clean debris along the gum line and to massage the gums. The dental pick is moved into a position where it can be used by twisting the dental flosser end of the handle back, which allows the dental floss pick to rotate forward.

### Advantages of the Invention

The most prominent advantages of the invention are:
1. Three dental cleaning features combined in one device: a dental flosser, tongue scraper and plastic floss pick.
2. An improved surface for tongue cleaning, a continuous sculpted edge that creates a ruffled edge, providing a comb-like effect for great efficacy.

### Comparison to Related Invention

This application is filed on the same day as the invention of the device Parallel to Face Sculpted Edge Tongue Scraper with Dental Flosser and Dental Floss Pick. Both devices rely on the same continuous sculpted edge which creates a ruffled effect to have a comb like effect on the tongue. The difference between the two are a different location for the continuous sculpted edge, this device has the continuous sculpted scraping edge on opposite end of the handle from the dental flosser. This device also has a cutout to help remove debris, and the dental floss pick rests against the handle until it is rotated into position. The Parallel to Face Sculpted Edge Tongue Scraper with Dental Flosser and Dental Floss Pick device has the continuous sculpted edge on the top edge of the handle, the dental flosser on one end of the handle, and the dental floss pick on the other edge of the handle,

### DRAWINGS—FIGURES

FIG. 1 is a ¾ view of the device, showing the sculpted edge along with the other features of the device. FIG. 1 shows the floss pick being rotated slightly away from the handle, the floss picks rotating due to the divots.

FIG. 2 shows the handle being flexed which allows the dental floss pick to move away from the handle.

FIG shows the handle tipped slightly down, it along with FIG. 4 show how the dental pick goes from side to side in the

FIG. 4 is a top view, with the handle tilted slightly upward, showing the other side of the handle from FIG. 3, of the device showing the sculpted edge and how it is a 90-degree angle from the handle.

FIG. 5 is a side view of the device, with the floss being held by the dental flossers prong facing out toward the viewer. Figure two illustrates how the continuous sculpted edge is at a 90-degree angle to the handle.

FIG. 6 is a head on view of the continuous sculpted tongue scraper, showing again the 90-degree angle of the tongue scraper to the handle. FIG. 6 has the bulk of the handle behind the continuous sculpted tongue scraper.

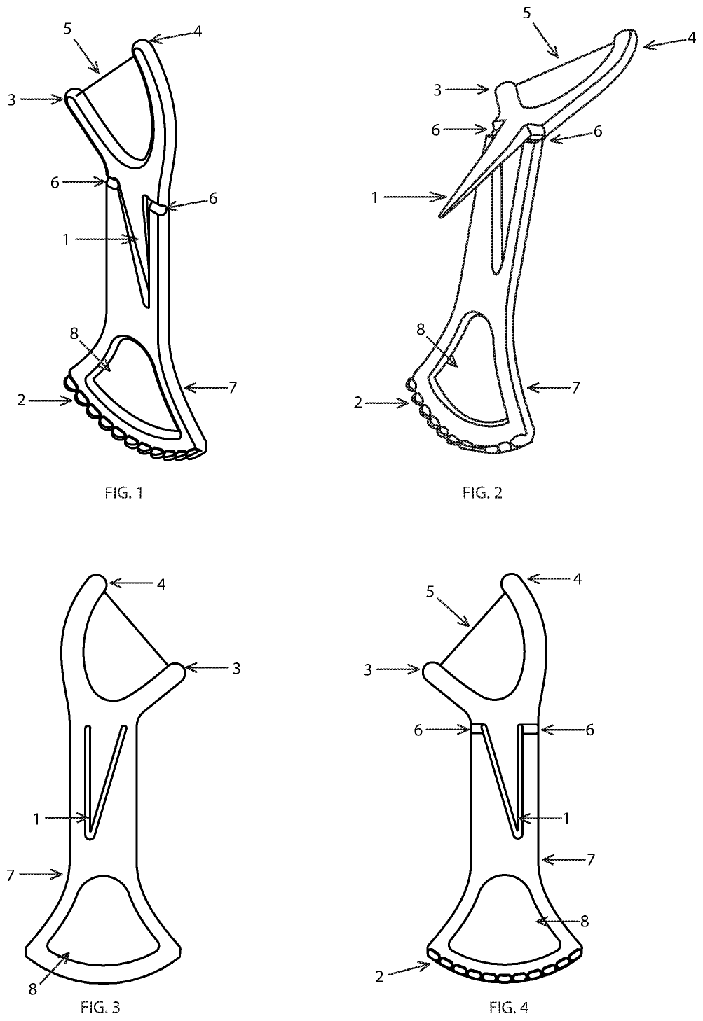

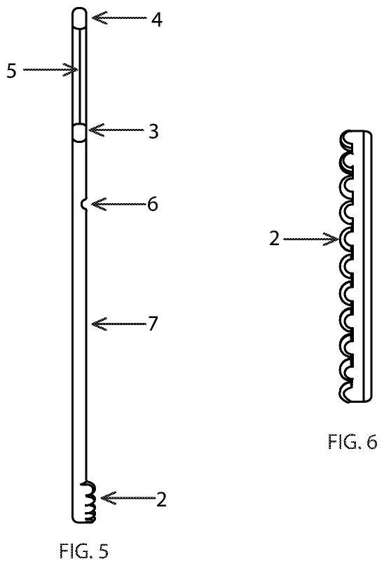

### DRAWINGS—REFERENCE NUMBERS

1. Dental floss pick.
2. Radiused continuous sculpted edge tongue scraper.
3. First tine of the dental flosser.
4. Second tine of the dental flosser.
5. Dental floss between the two tines
6. Divot to act as a holding crease for the dental floss pick
7. The handle.
8. Cutout near the end of the handle with the continuous sculpted edge tongue scraper,

### DETAILED DESCRIPTION OF THE INVENTION

The drawings present the preferred embodiment of the invention. Variations in each of the components is possible and mentioned in some of the additional embodiments listed in the SUMMARY OF THE INVENTION SECTION. FIG. 1 shows the preferred embodiment which comprises a handle 7, a radiused sculpted edge tongue scraper 2 on one end of the handle with the continuous sculpted edge at a 90 degree angle from the handle 7, two forked tines 3 and 4 that hold the dental floss 5, are both on one side of the end of the handle 7 opposite radiused sculpted edge tongue scraper 2. The dental floss pick 1 rests at the end of a tapered end of a triangle which moves away from the handle 7 when the handle 7 is flexed as with flexing the two divots 6 on of the dental floss pick allow the mechanism to pivot away from the handle 7. A cutout 8 near the end on the handle with the continuous sculpted edge tongue scraper 2 helps remove debris as it is cleaned by the continuous sculpted edge tongue scraper.

I claim:

1. A forward pulling continuous sculpted tongue scraper with a dental flosser and dental floss pick comprising:
a handle;
a radius sculpted edge at a 90 degree angle to one end of the handle, the sculpted portion covering the majority of the end of the handle, with the sculpted features of the handle continuous;
a tapered dental floss pick attached to the middle of the handle by a pair of divots, whereby the dental pick can move away from the handle when the handle is flexed;
a dental flosser with two tines in on the opposite end from the radius sculpted edge with the forks having a floss holding mechanism.

2. the tongue scraper of claim 1, wherein the sculpted features on one end of the handle are not continuous.

3. The tongue scraper of claim 1 wherein the dental flosser is at a 90-degree angle in relation to the handle.

4. The tongue scraper of claim 1 wherein a length of dental floss extends between the two tines of the dental flosser.
